(19) United States
(12) Patent Application Publication
(10) Pub. No.: US 2021/0077804 A1
(43) Pub. Date: Mar. 18, 2021

(54) STERILIZED VASCULAR ACCESS ASSEMBLIES AND RELATED KITS AND METHODS

(71) Applicant: CleanSite Medical, Inc., Solana Beach, CA (US)

(72) Inventors: John GRANT, Solana Beach, CA (US); Daniel M. CHAMBERS, Solana Beach, CA (US)

(73) Assignee: CleanSite Medical, Inc., Solana Beach, CA (US)

(21) Appl. No.: 17/026,163

(22) Filed: Sep. 18, 2020

Related U.S. Application Data
(60) Provisional application No. 62/902,389, filed on Sep. 18, 2019.

(51) Int. Cl. A61M 39/16; A61M 39/02

(52) U.S. Cl. CPC A61M 39/16; A61M 39/0247; A61M 2039/027; A61M 2039/0288; A61M 2039/0285

(57) ABSTRACT

This invention provides sterilized vascular access assemblies that include a sterilized needleless access connector (NAC) and a sterilized removable protective cap or cover capping or covering one or both of the NAC's fluid inlet and/or fluid outlet, and methods of making and using such assemblies, advantageously in the context of providing fluids, medication, and/or nutrition to patients in acute or long-term healthcare settings.

### RELATED APPLICATION

This application hereby claims the benefit of and priority to U.S. provisional patent application Ser. No. 62/902,389, filed 18 Sep. 2019, which is hereby incorporated by reference in its entirety for any and all purposes.

### TECHNICAL FIELD OF THE INVENTION

This invention is directed to single use caps for vascular access devices that have one or more ports for introducing fluids into a patient's vasculature, including, for example, vascular access devices such as needlefree, valved connectors as well as connectors where the access or injection site is adapted to receive a needle, and kits containing such devices and methods for using them.

### 1. INTRODUCTION

The following description includes information that may be useful in understanding the present invention. It is not an admission that any such information is prior art, or relevant, to the presently claimed inventions, or that any publication specifically or implicitly referenced is prior art.

### 2. BACKGROUND

Healthcare acquired infections (HAIs), i.e., infections that patients acquire while admitted to a hospital or surgery center for another, unrelated reason, are a primary concern in healthcare. Each year in the U.S., more than 100,000 people die from HAIs; many times that number become ill but do not die as a result of an HAI. Among the leading types of HAIs are catheter-related bloodstream infections (CRBSIs), which are infections that arise as a result of a patient having a central venous catheter and/or peripheral intravenous (IV) catheter inserted into a blood vessel, for example, a vein, to deliver hydration, nutrients, and/or medication.

As those in art appreciate, placement of a central line or peripheral IV compromises a patient's natural skin-based barrier against infection by microorganisms found in the environment, be they resident on the patient's skin, in the air, or on a surface that comes into contact with the patient. Indeed, an IV-caused compromise of a patient's skin is not just at the location where a catheter is inserted through the skin (the insertion site), but also at every opening that may be present on the fluid delivery set (typically comprised of a primary IV administration set and often a secondary extension set) used to connect one or more external fluid source(s) to the central line or peripheral IV catheter, as any such opening provides a potential entry point for pathogens into the patient's vasculature.

Prior to the outbreak of the AIDS epidemic in the 1980's, it was common practice to use needles to inject fluids and medicines directly into patients or, alternatively, into access or injection ports included in IV sets adapted to receive needles. When it became clear, however, that inadvertent needle-stick injuries were becoming a major source of HIV transmission between patients and healthcare workers, needlefree connectors (NCs) to provide needlefree (or needleless) vascular access were developed; their use has since become widespread. Indeed, in the U.S. today more than one billion NCs are used annually in healthcare settings in connection with providing peripheral venous and central line vascular access to patients in hospitals, outpatient surgery centers, dialysis centers, long-term care facilities, homes, and home-based healthcare facilities. Most NCs are used in IV sets, which may contain from as few as one to as many as 3, 4, 5, or more NCs. But because NCs serve as fluid access ports, they can also serve as access points for microbial pathogens. Indeed, widespread NC use in acute medicine has contributed to a marked increase in HAI incidence, particularly CRBSIs, because these devices rapidly become contaminated with microorganisms from the environment when they are removed from their sterile packaging and placed on a patient to deliver fluids to her/him as part of an IV set, for example.

As a result of such problems, in order to reduce the risk of infection from contaminated NCs, standard practice today includes a requirement that, before accessing an NC in an IV set, healthcare workers clean the NC surfaces in the IV's fluid flow path by scrubbing those surfaces with a sterile alcohol swab or wipe immediately prior to making a fluid connection to the valve, for example, by attaching a syringe to the valve to deliver a medication via a peripheral IV already connected to a patient. More recently, disinfecting caps (also called “port protectors”) have also been developed to provide up to 7 days of alcohol-based passive protection for NCs having luer fittings. Typically, such caps are threaded on to female or male luer fitting promptly after a healthcare worker first “scrubs the hub” of the fitting using an alcohol swab. Such a cap can be left on the connector for up to 7 days unless the connector is used before then to provide vascular access. Examples of such caps include Curos® (3M Corp.), SwabCap (ICU Medical, Inc.), and DualCap® (Merit Medical Systems). See, e.g., U.S. Pat. Nos. 7,780,794; 7,985,302; 8,206,514; 8,172,825; 8,523,831; 8,961,475; 9,114,915; 9,809,355; and 10,155,056. More recently, disinfecting caps provide active “scrubbing” capability in combination with up to 7 days of capping have also been developed. See, e.g., U.S. patent application Ser. No. 16/795,565.

Despite such advances, however, CRBSIs continue to represent a significant healthcare challenge. And given the magnitude of the mortality and morbidity associated with CRBSIs that result from peripheral IV and central line use, a long-recognized yet significant unmet need remains for articles or devices that can be used to reduce or eliminate the risk of initiating an HAI merely by accessing a patient's vasculature through an NC or other access or injection port adapted to receive a needle that is a component of an IV or other vascular access set connected to a catheter or cannula inserted into a blood vessel of a patient.

### 3. DEFINITIONS

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the singular forms “a”, “an”, and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

The terms “functional attachment”, “functional connection”, “functional reattachment”, “functional reconnection”, and the like refer to (re)attachment or (re)connection of or between two or more articles (e.g., a single use cap according to the invention and a needlefree connector) in an intended manner that allows each article to perform its intended function(s).

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The terms “removably attached”, “removably connected”, and the like refer to non-permanent attachment of connection between two or more articles, for example, between articles with complementary threaded regions, between articles in which one is press-fit (i.e., an interference fit between two parts in which one is forced under pressure into a slightly smaller hole, bore, cavity, etc. in the other) with another using complementary structures, etc.

A “single use cap” refers to a cap, cover, or protector for an access or injection port of a vascular access device that can only be used once; in other words, it can only be attached one time and removed one time from a vascular access device access or injection port because removal of the cap from the port physically alters at cap, or one or more components or sub-assemblies thereof, to as to prevent the cap from being reattached or reconnected to the same or a different vascular access device access or injection port in a way that would allow the reattached or reconnected cap to provide substantially the same degree of capping, covering, or protection to the port as compared to the initial attachment or connection.

The term “species”, when used in the context of describing a particular compound or molecule species, refers to a population of chemically indistinct molecules.

The term “vascular access port” means a port or valve on a vascular access device configured to provide fluid access to an IV set, catheter, cannula, or other device connected to, or designed for connection to, a patient in order deliver blood products (e.g., blood, plasma, etc.), hydration, nutrients, and/or medication.

### SUMMARY OF THE INVENTION

The object of the invention is to provide sterilized vascular access assemblies that include a sterilized needleless access connector (NAC) and a sterilized removable protective cap or cover capping or covering one or both of the NAC's fluid inlet and/or fluid outlet, and methods of making and using such assemblies, frequently in the context of providing fluids, medication, and/or nutrition to a patient in an acute or long-term healthcare setting.

Thus, one aspect of the invention relates to sterilized vascular access assemblies. Such assemblies include a NAC that defines a valved fluid flow path between a fluid inlet and a fluid outlet and either of (i) a removable protective cap or cover capping or covering the fluid inlet or fluid outlet or (ii) a first removable protective cap or cover capping or covering the fluid inlet and a second removable protective cap or cover capping or covering the fluid outlet. A removable protective cap or cover in such an assembly is configured to substantially maintain sterility of at least a portion of a surface that defines an exterior of the fluid inlet or fluid outlet of the sterilized needleless access connector until the cap or cover is removed from the corresponding fluid inlet or fluid outlet. Such assemblies are made by assembling the NAC, the removable protective cap(s) or cover(s), tubing, and other such fluid connectors and hardware (if any, depending on the particular assembly) as needed to complete the assembly, for example, an IV set, and extension set, etc. The assemblies are typically then packaged in a suitable container, for example, a sealed plastic bag (which may contain one or more vents), after which they are sterilized using a suitable sterilization technology, for example, gamma irradiation, e-beam exposure, or gas sterilization (e.g., using ethylene oxide).

In some embodiments, the sterilized vascular access assembly includes a removable protective cap that is threaded onto or into a corresponding structure on or in the fluid inlet or fluid outlet of the needleless access connector. In some of these embodiments, such as those that involve individually packaged and sterilized NACs intended for attachment to, for example, a central line catheter hub, an extension set attached to a peripheral IV catheter inserted into a peripheral vein of a patient, etc., both the fluid inlet and fluid outlet are capped and/or covered. Examples of fluid inlets and fluid outlets that can be capped or covered in accordance with the invention include male and female luer structures designed to matingly engage with corresponding structures (e.g., threads, thread tabs, luer tapers, etc.) on the removable protective cap.

In preferred embodiments, a removable protective cap or cover is configured to substantially maintain sterility the corresponding fluid inlet or fluid outlet to which the cap or cover is matingly engaged for a period of up to about 30 days under standard hospital room conditions, for example, 20° C.-25° C. at an atmospheric pressure of 1 atmosphere, after the assembly is removed from the sealed, sterilized packaging which the assembly is delivered to an end user. Frequently, one or more (e.g., 2-10) such assemblies are included as part of an IV set, an extension set, etc. sold to a hospital or other healthcare facility with the intention of being included as part of a vascular access system connected to a patient for the delivery of fluids, medication, and/or nutrition. Such systems can include, for example, a peripheral IV catheter inserted into a peripheral vein of a hospitalized patient, which catheter is then connected to an extension set which in turn is connected to an IV set that can then be connected to, for example, an IV bag. Delivery of fluids to a patient from such a system can be by any suitable method, including gravity flow or delivery controlled by an infusion pump.

A related aspect of the invention is directed to methods of maintaining the sterility of an external surface of a needleless access connector. Generally, such methods include configuring a sterilized vascular access assembly of the invention as part of a vascular access system that is connected to a patient and while leaving in place the cap or cover that caps or covers the fluid inlet of the needleless access connector until the needleless access connector is accessed for the first time. This can maintain the sterility of an external surface of the needleless access connector until first fluid access, i.e., until that particular NAC (often one of several) in the particular vascular access system is accessed by a nurse or other healthcare practitioner to deliver a fluid to the patient, for example, by injecting a liquid medication into the patient via the particular NAC and the vascular access system in which the NAC is included. As will be appreciated, some NACs in a vascular access system may be repeatedly, while others may never be accessed. Regardless, however, the invention allows the sterility of exterior surfaces of NACs that may be exposed to fluids being administered to a patient to be maintained until such time as the particular NAC is to be accessed for the first time. At that point a healthcare worker removes the removable protective cap or cover and then makes the intended fluid connection.

Another aspect of the invention concerns methods of reducing healthcare-associated infection (HAI) risk. These methods involve configuring a sterilized vascular access assembly of the invention as part of a vascular access system connected to a patient and leaving in place the cap or cover that caps or covers the fluid inlet of the needleless access connector, thereby reducing the risk that the patient will acquire an HAI.

Still another aspect of the invention addresses methods of enhancing patient safety. Such methods typically involve configuring a sterilized vascular access assembly of the invention as part of a vascular access system connected to a patient and leaving in place the cap or cover that caps or covers the fluid inlet of the needleless access connector, thereby enhancing patient safety by reducing a risk that the patient will acquire an HAI.

### BRIEF DESCRIPTION OF THE DRAWINGS

Unless otherwise indicated, it is understood that the drawings are not to scale, as they are intended merely to facilitate understanding of the invention as opposed to specific dimensions, etc. In the drawings, like numbers in two or more drawings represent like elements. The illustrative embodiments described herein are not meant to be limiting. Other embodiments may be utilized, and other changes may be made, without departing from the spirit or scope of the subject matter presented here.

FIG. 1 depicts representative sterilized vascular access assemblies according to the invention.

### DETAILED DESCRIPTION OF THE INVENTION

This invention concerns sterilized vascular access assemblies that include a sterilized needleless access connector (NAC) and a sterilized removable protective cap or cover capping or covering one or both of the NAC's fluid inlet and/or fluid outlet. Needleless access connectors play an important role in healthcare today as they serve to reduce the risk of inadvertent needle-stick injuries, particularly to healthcare workers. At least 1 billion such connectors are used annually in the U.S. in connection with providing peripheral venous and central line vascular access to patients. But because NACs serve as fluid access ports, they can also serve as access points for microbial pathogens. Indeed, they are known to be among the primary sources for catheter-related bloodstream infections, which result in significant morbidity and mortality among patients who receive central lines and/or peripheral IVs in connection with their medical treatments.

Here, a “cap” refers to a device that covers, protects, or shields at least part of a cylindrical portion of another device, such as the threaded male or female portion of a luer fitting on the fluid inlet or fluid outlet portion of a NAC. In the context of capping the fluid inlet portion of a NAC, which contains the part of the NAC's valve configured to be engaged by a complementary fitting to actuate the valve so that fluid can flow into and through the NAC via its engineered fluid pathway, a cap will, when matingly suitably engaged with the fluid inlet portion of the NAC, not only protect the accessible valve surface but also at least some (about 10% or more) of the threaded sidewall of the fluid inlet portion of the NAC. In some preferred embodiments, a cap covers all of the threaded region of a NAC's fluid inlet portion. A “cover” refers to a device that covers, protects, or shields the top portion of the cylindrical end of a NAC and not more than about 10% or less of the adjacent sidewall.

Each sterilized removable protective cap or cover is configured to substantially maintain sterility of at least a portion of a surface that defines an exterior of the fluid inlet or fluid outlet of the sterilized needleless access connector until the cap or cover is removed from the corresponding fluid inlet or fluid outlet.

Here, to “substantially maintain sterility” of a surface means to provide protection over time for a surface such that the surface is less susceptible to microbial contamination by a factor of 2, 5, 10, 100, 1,000, 10,000, 100,000, 1,000,000, 10,000,000, 100,000,000, or a billion, 10 billion, 100 billion, 1 trillion or more, as compared to an equivalent surface that has not been so protected. Such time periods can range from, for example, about 1 hour to about 30 days under standard hospital room conditions (20° C.-25° C. and an atmospheric pressure of 1 atmosphere) after removal from a sealed, sterilized package. In addition to serving to substantially maintain sterility, the caps and/or covers used to cap or cover NAC fluid inlets in the assemblies of the invention will also serve to prevent inadvertent or accidental actuation of the valve portion of the protected NAC, thereby reducing the risk that one or more contaminating microorganisms may gain entry into NAC's fluid pathway and thus potentially into the patient's vasculature and thereby possibly cause an HAI.

In embodiments that employ caps, the caps preferably thread onto or into a corresponding structure on or in the fluid inlet or fluid outlet of the sterilized needleless access connector to which the cap is attached. Examples of preferred threaded configurations include male and female luer fittings. In embodiments where one or more sterilized removable protective covers are used, attachment may be by way of adhesive, press fit, or any other suitable approach.

Preferably, sterilized vascular access assemblies of the invention are packaged in sealed, sterilized packaging, with or without labels, instructions for use, and the like. Sterilization of the devices, assemblies, kits, and packages of the invention can be accomplished by any suitable sterilization method compatible with the components to be sterilized. Preferred methods include gamma or electron beam irradiation. Preferably, sterilization of an assembly occurs after it has been suitable packaged.

As will be appreciated, the sterilized vascular access assemblies of the invention are designed for use in connection with providing vascular access to patients. Typically this is by way of inclusion in a vascular access system (e.g., an IV bag, IV set, extension set, and IV catheter inserted into a peripheral vein) connected to a patient to provide fluids, medication, and/or nutrition to the patient. Other vascular access systems include central lines, including peripherally inserted central lines.

In many preferred embodiments, the sterilized vascular access assemblies of the invention are included as part of a sterilized vascular access extension set and/or sterilized IV set.

### Representative Embodiments of the Invention

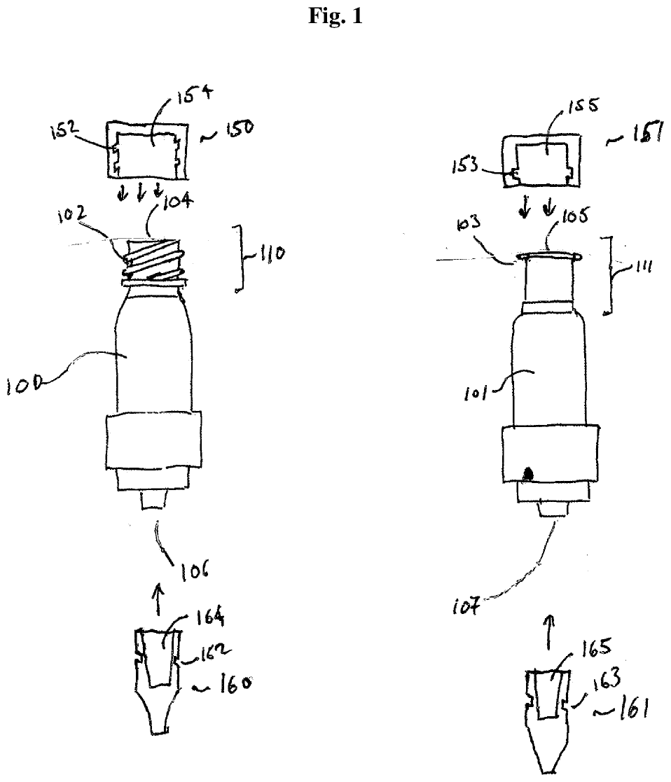

FIG. 1 shows two representative sterilized vascular access assemblies according to the invention.

The embodiment shown on the left shows a NAC (100) that defines a valved fluid flow path between a fluid inlet (104) and a fluid outlet (106). The NAC includes a valve region (110) that has threads (102) to facilitate connection to other components that have complementary threaded regions. The assembly also includes a removable first protective cap (150) for capping or covering the NAC's fluid inlet (104) and a removable second protective cap (160) for capping the fluid outlet (106). The removable first protective cap (150) includes complementary threads (152) to the threads (102) on the NAC's valve region (110). The removable first protective cap (150) defines a cavity (154) designed to accept the NAC's valve region (110) so as to cap, protect, and maintain the sterility of the NAC's valve region (110) after the assembly has been assembled and sterilized. The assembly's second protective cap (160) defines a cavity (164) designed to accept the NAC's male luer taper so as to cap, protect, and maintain the sterility of the male luer taper after the assembly has been assembled and sterilized.

The embodiment shown on the left shows a NAC (101) that defines a valved fluid flow path between a fluid inlet (105) and a fluid outlet (107). The NAC includes a valve region (111) that has thread tabs (103) to facilitate connection to other components that have a complementary threaded region. The assembly also includes a removable first protective cap (151) for capping or covering the NAC's fluid inlet (105) and a removable second protective cap (161) for capping the fluid outlet (107). The removable first protective cap (151) includes complementary thread tab acceptors (153) to the thread tabs (103) on the NAC's valve region (111). The removable first protective cap (151) defines a cavity (155) designed to accept the NAC's valve region (111) so as to cap, protect, and maintain the sterility of the NAC's valve region (111) after the assembly has been assembled and sterilized. The assembly's second protective cap (161) defines a cavity (166) designed to accept the NAC's male luer taper so as to cap, protect, and maintain the sterility of the male luer taper after the assembly has been assembled and sterilized.

Unless the context clearly requires otherwise, throughout the description above and the appended claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list, and any combination of the items in the list.

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law.

Any patent or patent application referenced herein is hereby incorporated by reference in its entirety.

We claim:

1. A sterilized vascular access assembly, comprising:
a. a needleless access connector that defines a valved fluid flow path between a fluid inlet and a fluid outlet; and
b(i). a removable protective cap or cover capping or covering the fluid inlet or fluid outlet, or
b(ii). a first removable protective cap or cover capping or covering the fluid inlet and a second removable protective cap or cover capping or covering the fluid outlet,
wherein each removable protective cap or cover is configured to substantially maintain sterility of at least a portion of a surface that defines an exterior of the fluid inlet or fluid outlet of the sterilized needleless access connector until the cap or cover is removed from the corresponding fluid inlet or fluid outlet, and
wherein the assembly is sterilized.

2. A sterilized vascular access assembly according to claim 1 that comprises a removable protective cap threaded onto or into a corresponding structure on or in the fluid inlet or fluid outlet of the needleless access connector.

3. A sterilized vascular access assembly according to claim 2 wherein the fluid inlet and/or fluid outlet comprises a male or female luer structure matingly engaged with the removable protective cap.

4. A sterilized vascular access assembly according to claim 1 packaged in a sealed, sterilized package.

5. A sterilized vascular access assembly according to claim 4 wherein any removable protective cap or cover in the assembly is configured to substantially maintain sterility the corresponding fluid inlet or fluid outlet to which the cap or cover is matingly engaged for a period of up to about 30 days under standard hospital room conditions (20° C.-25° C. and an atmospheric pressure of 1 atmosphere) after removal from a sealed, sterilized package.

6. A sterilized vascular access assembly according to claim 1 disposed in a vascular access system connected to a patient to provide fluids, medication, and/or nutrition.

7. A sterilized vascular access extension set comprising a sterilized vascular access assembly according to claim 1.

8. A sterilized vascular access extension set according to claim 7 packaged in a sealed, sterilized package.

9. A sterilized vascular access extension set according to claim 7 disposed in a vascular access system connected to a patient.

10. A sterilized IV set comprising at least one sterilized vascular access assembly according to claim 1.

11. A sterilized IV set according to claim 10 packaged in a sealed, sterilized package.

12. A sterilized IV set according to claim 10 disposed in a vascular access system connected to a patient.

13. A method of maintaining sterility of an external surface of a needleless access connector, comprising configuring a sterilized vascular access assembly according to claim 1 as part of a vascular access system connected to a patient and leaving in place the cap or cover that caps or covers the fluid inlet of the needleless access connector until the needleless access connector is accessed for the first time, thereby maintaining sterility of an external surface of the needleless access connector until first fluid access.

14. A method of reducing healthcare-associated infection (HAI) risk, comprising configuring a sterilized vascular access assembly according to claim 1 as part of a vascular access system connected to a patient and leaving in place the cap or cover that caps or covers the fluid inlet of the needleless access connector, thereby reducing the risk that the patient will acquire an HAI.

15. A method of enhancing patient safety, comprising configuring a sterilized vascular access assembly according to claim 1 as part of a vascular access system connected to a patient and leaving in place the cap or cover that caps or covers the fluid inlet of the needleless access connector, thereby enhancing patient safety by reducing a risk that the patient will acquire an healthcare-associated infection (HAI).
